(19) United States
(12) Patent Application Publication
(10) Pub. No.: US 2002/0107537 A1
(43) Pub. Date: Aug. 8, 2002

(54) Metal breath freshening device

(76) Inventor: Lucia Singh, Saline, MI (US)

Correspondence Address: LUCIA SINGH, 9880 YORK WOODS DR, SALINE, MI, 48176, US

(21) Appl. No.: 09/735,420

(22) Filed: Dec. 12, 2000

(51) Int. Cl. A61B 17/24

(52) U.S. Cl. 606/161

(57) ABSTRACT

An elongated article comprising a handle attached to a bulbous, metal-covered head. The article is used to freshen the breath by swabbing the inside of the mouth with the head. The end of the handle opposite of the head may be flattened and ridged to facilitate scraping of the tongue.

### BACKGROUND

1. Field of Invention

This invention relates to breath freshening devices.

2. Prior Art

Various devices and products have been devised for cleansing the mouth and freshening the breath, notably the toothbrush, dental floss, mouthwash, aromatic candies, and toothpick. However, each of these as disadvantages.

The toothbrush is probably the most widely used breath-freshening device. It is generally effective, but suffers from some disadvantages. The bristles wear out and splay after some use, thus requiring periodic replacement of the toothbrush. The use of the toothbrush requires rinsing the mouth with water, thus restricting the use of the toothbrush near a sink. The toothbrush is commonly used with dentifrice, which must be periodically replaced. After use, the toothbrush is wet, and the bristles hold water, thus making the toothbrush inconvenient to carry in a pocket or purse. In addition, the toothbrush can hold and accumulate food particles in its bristles, thus requiring it to be thoroughly cleaned after each use. Furthermore, the toothbrush also may not help to reduce certain food odors, particularly if used without dentifrice.

Dental floss, while effective in removing food particles lodged between the teeth, has the disadvantage of being consumable. After one use, the piece of floss is usually discarded. Also, use of dental floss can be painful, especially for first-time users. Furthermore, use of dental floss normally requires both hands and proximity to a sink for expectorating and a garbage can to discard the used piece of floss, thus the use of dental floss is restrictive to certain locations. Also, dental floss may not be effective for removing certain food odors.

Like dental floss, mouthwash also has the disadvantages of being consumable and restrictive to certain locations. Breath-freshening candies also are consumable. They also have the further disadvantage of merely masking the odor, and not actually removing or breaking down odor-causing particles. Furthermore, some breath-freshening candies, because of their sugar content, may promote tooth decay.

Finally toothpicks are sometimes used to freshen the breath, but they also have some of the same problems as those mentioned above. In addition, if the pick is sharp, it may be dangerous.

Therefore, the need exists for a device and method of freshening the breath that is safe, portable, non-consumable, easily cleaned, and effective.

My present invention is based on a totally different operating principle than the products listed above. The operating principle of my invention is not used by any known product to clean the mouth, or freshen the breath, but rather by a product trademarked ODOR STEELER, owned by DUOTRADE TRADING LDA. CORPORATION PORTUGAL, sold to clean the hands. The ODOR STEELER bar is a teardrop shaped bar about three inches long made of stainless steel. By simply rubbing one's hands with the bar under water, odors from poultry, garlic, onion, fish and other foods are removed. The product does not require soap or scrubbing. It does not wear out and is easy to clean. It does not require batteries or electrical power to operate. It is safe and nontoxic. It can be manufactured cheaply and sold at a profit.

No product currently sold to clean the mouth operates on the principle used by the ODOR STEELER bar to clean the hands. Nor does any product currently sold have the advantages of being able to remove offensive odors of poultry, garlic, onion, fish and other foods while being non-consumable, durable, easy to clean, and profitable.

### SUMMARY

In accordance to the present invention, a breath freshener comprises a handle attached to a head having a portion covered with metal.

Objects and Advantages

Accordingly, several objects and advantages of my breath freshener are:

(a) to provide a breath freshener which effectively removes the odors of poultry, garlic, onion, fish and other foods from the surfaces of the mouth,

(b) to provide a breath freshener which can be used without dentifrice or other consumable product including electricity,

(c) to provide a breath freshener which normally does not require replacement, but rather is durable enough to work well even after considerable use,

(d) to provide a breath freshener which is easily cleaned and dried,

(e) to provide a breath freshener which can be conveniently carried in a pocket or purse,

(f) to provide a breath freshener which is easy to use and painless, even for inexperienced users,

(g) to provide a breath freshener which will not promote tooth-decay,

(h) to provide a breath freshener which is can be comfortably used in situations where it is not appropriate to use a toothbrush or dental floss, and

(i) to provide a breath freshener which can be manufactured and sold profitably.

These and other objects and features of the present invention will become more fully apparent from the following description and appended claims, or may be learned by the practice of the invention as set forth hereinafter.

### BRIEF DESCRIPTION OF DRAWINGS

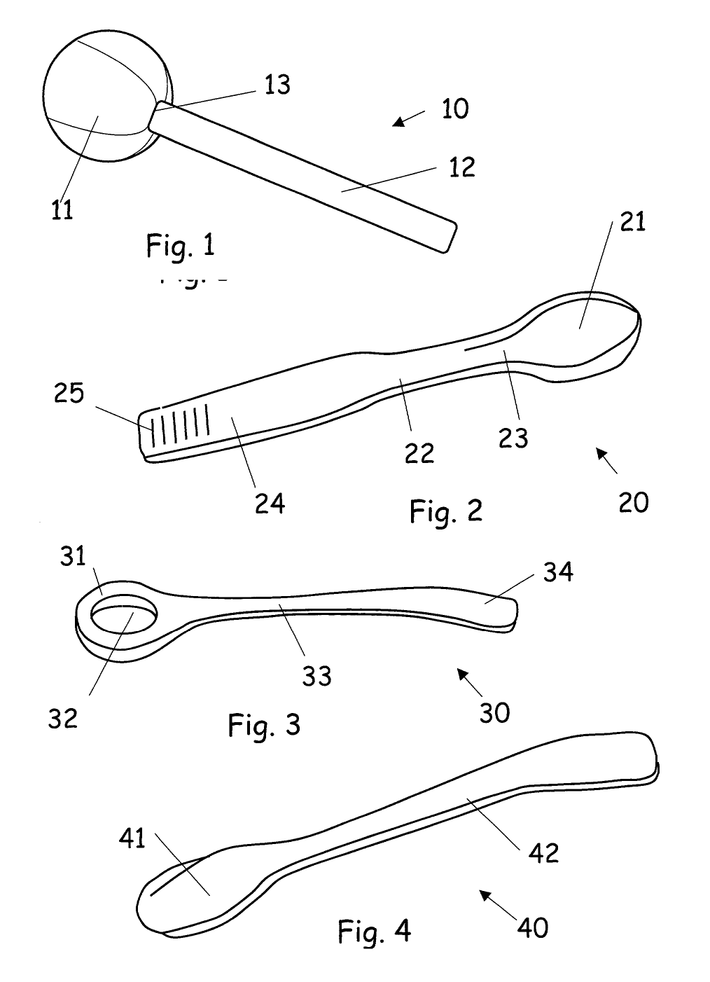

The accompanying FIGS. 1-4 show a first, second, third, and fourth embodiment of my breath freshener respectively.

| | |
|---|---|
| 10 First embodiment | 11 spherical head |
| 12 handle | 13 attachment of head and handle |
| 20 Second embodiment | 21 bulbous head |
| 22 handle | 23 attachment of head and handle |
| 24 flattened portion of handle | 25 ridged or serrated portion of handle |
| 30 Third embodiment | 31 annular head |
| 32 hole in head | 33 handle |
| 34 flattened portion of handle | 40 Fourth embodiment |
| 41 flattened head | 42 handle |

### DESCRIPTION

First Embodiment

FIG. 1 shows a perspective view of first preferred embodiment 10 of my breath freshener. Head 11 is shaped so as to be comfortable to hold in the mouth and is covered with metal. In embodiment 10, head 11 is spherical in shape with a radius of about 12 mm. However, the exact size and shape of the head may vary according to size and shape of the mouth to be cleaned. For example, the shape of the head can be ellipsoidal, bulbous, annular, or convex. In addition to such common shapes, the head may be specially shaped to accommodate hard-to-reach areas of the mouth or irregularly shaped areas. Furthermore, the head may be shaped according to stylistic or marketing considerations. For example, the head may be shaped like an actual human or animal head to attract children.

The surface of head 11 is smooth metal, preferably stainless steel because stainless steel has the required cleansing effect, is nontoxic, does not rust, and is relatively inexpensive. Alternatively, other suitable metals can be substituted. In the preferred embodiment, the entire surface of the head is covered with metal. This is because it is the metal that causes the breath freshening, or cleaning, effect. Furthermore, metal is easy to clean. However, in order to reduce costs, or for aesthetic reasons, some portions of the head may be non-metal. Although the preferred embodiments feature smooth metal heads, alternative embodiments may employ textured or undulating surfaces to increase the effective surface area of the metal or to provide a more abrasive surface.

Head 11 can be solid metal, or hollow, or formed from some non-metal material and then simply surfaced with metal. The method of constructing the head can vary according to the influences of cost, available technology, style, desired weight, durability and other marketing considerations. In order for my invention to function properly, however, it is important that at least some portion of the surface of the head be metal.

In embodiment 10, handle 12 is cylindrical, approximately 7 mm in diameter and between 7 and 15 cm long. Although handle 12 is pictured as straight and simple cylinder, the shape, size, and composition of the handle may differ so as to be more stylish or comfortable to grasp, hold or to reach into the mouth. The handle can be made of the same material as the head, or another suitable nontoxic material may be used such as wood or plastic.

Handle 12 is attached to head 11 at attachment 13. The exact means of attachment can vary. Attachment may be permanent, such as if head 11 and handle 12 are one integral piece of metal, or temporary, such as if handle 12 is threaded into head 11. A removable head may present advantages by allowing different sized and shaped heads to be interchanged. The choice of exactly how head 11 is attached to handle 13 is a design choice influenced by marketing considerations such as those listed above.

Operation

To use my invention, a user grasps the handle 12 and inserts head 11 into her mouth, swabbing and rubbing head 11 against the cheeks, gums, palate, tongue and other parts of her mouth. If the handle has a portion on it for tongue cleaning, for example as shown in FIG. 2 at 24 and 25, then that portion of the handle may also be used to rub and scrape the tongue.

Though not the primary aim of my present invention, my breath freshener may also be used by one person to clean the mouth of another person not holding the handle, such as a disabled person.

Alternatively, the mouth of an animal such as a cooperative dog or cat may be cleaned. In such a case, the head would have to be sized and shaped accordingly.

Other embodiments

FIG. 2 depicts a second preferred embodiment 20 of my invention. In this embodiment, head 21 and handle 22 merge to form a smooth piece of metal. Embodiment 20 is preferably formed from a single, solid piece of stainless steel.

Head 21 is a rounded, smooth, three-dimensionally convex or bulbous shape. Head 21 is designed to be comfortably held in the mouths; it has rounded edges. Head 21 merges smoothly into handle 22 at attachment 23 so that the whole apparatus can be easily cleaned and accumulation of dirt and filth avoided. The end of the handle 24 is flattened so that it can be used to rub the tongue. Surface roughening means 25 such as ridges, serrations, striations or texture 25 may be included to facilitate scraping.

In third embodiment 30 depicted in FIG. 3, head 31 is annular or doughnut-shaped. Hole 32, by reducing the mass of the head, may make the head more comfortable to hold in the mouth for some users.

In fourth embodiment 40 of my invention depicted in FIG. 4, head 41 is a flattened, three-dimensionally convex shape merging smoothly into slightly bent handle 42.

Manufacture

My breath freshener can be manufactured using techniques known to those skilled in the arts of making similarly sized articles such as kitchen and eating utensils, metal tools, or toothbrushes.

The head and handle can be formed from a single piece, similar to how one-piece, solid-metal flatware is manufactured. Alternatively, the head and handle can be manufactured separately and then attached using glue, welding, pressure, screws and other means known to those skilled in the art. The attachment may or may not be permanent. A removable head would allow for its repair or replacement.

My breath freshener can be constructed of solid metal. Alternatively, parts of my breath freshener may be hollow or filled with other material so as to lessen weight, decrease manufacturing costs, or improve appearance. At least some portion of the surface of the head needs to be metal, because the breath-freshening effect depends on the metal.

Theory of Operation

While I believe that my breath freshener operates by increasing the flow of saliva and facilitating the breakdown of odor-causing molecules by a catalytic effect of the metal, I don't wish to be bound by this. I believe that my breath freshener works on the mouth similarly to how the ODOR STEELER bar works on the hands. In both cases, a metal surface is rubbed against the skin in the presence of water. In the case of the ODOR STEELER bar, the water comes from a tap; in the present case, the water comes from the saliva generated by the mouth.

Advantages

From the above description, certain advantages of my breath freshener become evident: When formed from a single, solid piece of stainless steel, as shown in FIG. 2, my breath freshener is very durable and easy to clean and dry. There are no bristles or other moving parts to wear out or break. The smooth, broadly curved surfaces do not hold dirt or water. In addition, it can be inexpensively mass-produced using the same technology as that used to make solid flatware.

Furthermore, my breath freshener is extremely convenient. Because it is self-contained and does not requiring any additional products to be used with it, there is nothing more for a user to buy. It can be used while sitting at the office, driving, or walking. After use, it can be simply wiped or rinsed and put in a pocket or purse.

Conclusion, Ramifications, and Scope of My Invention

Thus the reader can see the advantages of my invention. My breath freshener is easy to use, inexpensive to produce, and effective for reducing and eliminating bad breath. In addition, my breath freshener is sturdy and long lasting. It requires little maintenance, does not wear out, and does not require additional products to operate. In addition, my breath freshener is portable and does not require a power supply such as a battery.

While my above description contain many specifics, they should not be construed as limitations on the scope of my invention, but rather as exemplifications of preferred embodiments thereof. Many other variations are possible. For example, the head can have many different and specially shaped configurations to accommodate different sized mouths or hard-toread areas of the mouth. Also, the handle can mimic the handle shape of any of the multitude of toothbrushes available.

Accordingly, the scope of the invention should be determined not by the embodiments illustrated, but by the appended claims and their legal equivalents.

I claim:

1. A manufactured article for cleaning the mouth and freshening the breath of a person comprising:
a) head means for cleaning the mouth and freshening the breath of said person, wherein said head means are sized and shaped to comfortably fit inside said person's mouth and wherein a portion of the surface of said head means is metal,
b) handle means for enabling said person to grasp said article, and
c) attachment means attaching said head means to said handle means.

2. The article of claim 1 wherein said metal is stainless steel.

3. The article of claim 1 wherein said handle means has a flattened portion for cleaning the tongue of said person.

4. The article of claim 3 wherein said flattened portion has surface roughening means for facilitating the cleaning of the tongue of said person.

5. The article of claim 1 wherein the shape of said head means is one selected from the group consisting of: spherical, ellipsoidal, bulbous, annular, or convex.

6. The article of claim 1 wherein said head means are specially shaped to facilitate the reaching of certain areas of said mouth.

7. The article of claim 1 wherein the surface of said article is smooth so as to prevent accumulation of dirt and to facilitate cleaning of said article.

8. A process of freshening a person's breath comprising the steps of:
(a) providing an elongated article at least partially covered with metal,
(b) rubbing the inside of said person's mouth with the metal-covered portion of said article.

9. The process of FIG. 8 wherein said elongated article comprises handle means and head means wherein said handle means are attached to said head means and said metal-covered portion includes the head means.

10. An oral cleaning apparatus for cleaning the mouth of odor-causing substances from a user's mouth comprising:
(a) a handle,
(b) a head having a portion covered with metal,
(c) connecting means attaching said handle to said head, wherein said handle can be grasped to swab the inner surfaces of the mouth with said head.

11. The apparatus of claim 10 wherein said metal is stainless steel.

12. The apparatus of claim 10 wherein said handle is attached to said head at one end and the other, opposite end of said handle is flattened.

13. The apparatus of claim 12 wherein said opposite end of said handle is striated or ridged to allow said user to scrub the tongue.

14. The apparatus of claim 11 wherein said head has a substantially spherical shape.

15. The apparatus of claim 11 wherein said head has a substantially annular shape.

16. The apparatus of claim 11 wherein said head has a substantially convex shape.

17. The apparatus of claim 11 wherein said head is specially shaped to facilitate the reaching of areas of the mouth.
